(19) United States
(12) Patent Application Publication
(10) Pub. No.: US 2026/0367203 A1
(43) Pub. Date: Nov. 19, 2026

(54) SCANNING A K-SPACE USING DIFFERENT RADIO FREQUENCY TRANSMISSION PULSES

(71) Applicant: Siemens Healthineers AG, Forchheim (DE)

(72) Inventor: Patrick Liebig, Erlangen (DE)

(21) Appl. No.: 19/679,386

(22) Filed: May 15, 2026

(30) Foreign Application Priority Data
May 15, 2025 (DE) 10 2025 118 838.6

(51) Int. Cl. G01R 33/48; A61B 5/055; G01R 33/56

(52) U.S. Cl. CPC G01R 33/4818; G01R 33/5608; A61B 5/055

(57) ABSTRACT

A method for scanning a k-space during magnetic resonance measurement of a patient and a magnetic resonance apparatus are provided. The method includes providing a first radio frequency transmission pulse for generating first magnetic resonance signals, providing a second radio frequency transmission pulse for generating second magnetic resonance signals, segmenting the k-space to be scanned into at least one first region and at least one second region, scanning the at least one first region of the k-space using the first radio frequency transmission pulse and scanning the at least one second region of the k-space using the second radio frequency transmission pulse.

This application claims the benefit of German Patent Application No. DE 10 2025 118 838.6, filed on May 15, 2025, which is hereby incorporated by reference in its entirety.

### BACKGROUND

The present embodiments relate to a method for scanning a k-space during magnetic resonance measurement of a patient and a magnetic resonance apparatus.

In medical technology, imaging using magnetic resonance (MR), also referred to as Magnetic Resonance Imaging (MRI), is characterized by high soft tissue contrast. In the process, a patient is positioned in the examination area of a magnetic resonance apparatus in which a main magnet of the magnetic resonance apparatus generates a main magnetic field (also referred to as a B0 field). During a magnetic resonance measurement, radio frequency (RF) transmission pulses (e.g., radio frequency transmission pulses) to generate an alternating magnetic field (also referred to as a B1 field) and gradient pulses to generate a magnetic field gradient superimposed on the main magnetic field are transmitted into the examination area according to a magnetic resonance sequence, and nuclear spins are thus excited in the patient. As a result, spatially encoded magnetic resonance signals are triggered in the patient. The magnetic resonance signals may be used to reconstruct magnetic resonance images.

The amount of radio frequency energy absorbed per unit of time and per kilogram of body weight of the patient following the transmission of radio frequency transmission pulses may be referred to as the specific absorption rate (SAR). The absorption of RF energy may result in heating of the body tissue of the patient, or even to burns. To ensure the safety of the patient, the SAR is usually limited (e.g., the SAR must not exceed a certain threshold value). To prevent this threshold value from being exceeded during the execution of a magnetic resonance sequence, the measurement is, for example, terminated, the number of slices to be measured is reduced, and/or the measurement time is extended.

Unfortunately, radio frequency transmission pulses with which magnetic resonance signals may be generated to reconstruct (e.g., excite) magnetic resonance images of particularly high quality often result in high SAR exposure of the patient. For example, parallel radio frequency transmission pulses (pTx pulses) may generate a homogeneous B1 field through simultaneous transmission using multiple transmitter coils that, however, may also result in high SAR exposure. A compromise may therefore be made with regard to image quality (e.g., signal homogeneity) and the energy input into the patient. A magnetic resonance sequence in which high-quality magnetic resonance images may be generated with low patient exposure would be desirable.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, a magnetic resonance sequence with which high-quality magnetic resonance images may be generated with minimal patient discomfort is provided.

Accordingly, a computer-implemented method for scanning a k-space during magnetic resonance measurement of a patient is provided. In this method, a first radio frequency transmission pulse for generating first magnetic resonance signals is provided. Further, a second radio frequency transmission pulse for generating second magnetic resonance signals is provided.

The generation of the first and/or second magnetic resonance signals may involve various aspects of the generation of magnetic resonance signals (e.g., excitation and/or refocusing and/or saturation). The first and/or second radio frequency transmission pulse may thus, for example, be an excitation pulse and/or a refocusing pulse and/or a saturation pulse.

A k-space to be scanned is at least segmented into at least one first region and at least one second region (e.g., the k-space to be scanned is segmented into at least one or more first regions and one or more second regions). The at least one first region of the k-space is scanned using the first radio frequency transmission pulse (e.g., the one or more first regions of the k-space are scanned using the first radio frequency transmission pulse). The at least one second region of the k-space is scanned using the second radio frequency transmission pulse (e.g., the one or more second regions of the k-space are scanned using the first radio frequency transmission pulse). In addition to the at least one first region and the at least one second region, the k-space may have further regions, or the k-space may consist only of the at least one first region and the at least one second region.

The first and/or second radio frequency transmission pulse may, for example, be provided in the form of a dataset (e.g., an electronic dataset). The first radio frequency transmission pulse and/or the second radio frequency transmission pulse may, for example, be loaded from a database and/or calculated.

The first radio frequency transmission pulse and/or the second radio frequency transmission pulse may be part of a magnetic resonance sequence using which k-space is scanned. A wide variety of magnetic resonance sequences may be provided. The magnetic resonance sequence may, for example, be echo planar imaging (EPI) sequence (e.g., a readout segmentation of long variable echo (RESOLVE) sequence (e.g., RESOLVE trains). The scanning of the at least one first region of the k-space and the at least one second region of the k-space may be part of a magnetic resonance measurement of the patient.

Segmenting a k-space to be scanned into at least one first region and one second region may, for example, involve dividing the k-space to be scanned into the at least one first region and into the at least one second region. For example, the k-space to be scanned may be segmented or divided into N first regions and M second regions, N and M being integers with N≥1 and M≥1.

The at least one first region and/or the at least one second region of the k-space to be scanned may each include multiple k-space points and/or k-space lines (e.g., k-space rows). Multiple, adjacent k-space points and/or k-space lines may be combined into one region.

The k-space may, for example, be a two-dimensional k-space or a three-dimensional k-space. Accordingly, a region of a two-dimensional k-space may, for example, represent a surface of the k-space. Accordingly, a region of a three-dimensional k-space may, for example, represent a volume of the k-space.

For example, k-space may be a two-or three-dimensional data model derived from digitized raw data for a measured image. The k-space may be the same as the raw data matrix that is filled in during a scan of the k-space (e.g., with lines, such as rows). The axes of the k-space may, for example, be designated kx (e.g., horizontal axis) and ky (e.g., vertical axis). The data points of the plane lying within the axes of the k-space may, for example, represent spatial frequencies. The spatial frequency data may be converted into a magnetic resonance image (e.g., using a Fourier transform).

Scanning the at least one first region of k-space using the first radio frequency transmission pulse may involve transmitting the first radio frequency transmission pulse at least once (e.g., multiple times). Scanning the at least one second region of the k-space using the second radio frequency transmission pulse may include transmitting the second radio frequency transmission pulse at least once (e.g., multiple times).

The first and/or second radio frequency transmission pulse may, for example, be transmitted by a radio frequency antenna unit of the magnetic resonance apparatus. The radio frequency antenna unit may include one or more transmitter coils for this purpose. The first and/or second radio frequency transmission pulse may, for example, be amplified by one or more radio frequency amplifiers and radiated into the patient by the one or more transmitter coils.

The scanning of a region may, for example, be performed in a number of sections, one radio frequency transmission pulse being transmitted for the scanning of each section. To scan the at least one first region of the k-space using the first radio frequency transmission pulse, the first radio frequency transmission pulse may, for example, be transmitted multiple times. A magnetic resonance sequence for scanning the at least one first region of the k-space may include multiple first radio frequency transmission pulses. To scan the at least one second region of the k-space using the second radio frequency transmission pulse, the first radio frequency transmission pulse may, for example, be transmitted multiple times. A magnetic resonance sequence for scanning the at least one second region of k-space may include multiple second radio frequency transmission pulses.

If the scanning of a region takes place in a number of sections, these sections may, for example, be scanned in rapid succession or distributed throughout the magnetic resonance sequence.

By scanning the at least one first region of k-space, for example, the first magnetic resonance signals are generated (e.g., excited). By scanning the at least one second region of k-space, for example, the second magnetic resonance signals are generated (e.g., excited). In one embodiment, the first and/or second generated magnetic resonance signals are received (e.g., acquired and/or recorded) by the magnetic resonance apparatus (e.g., by receiver coils of the magnetic resonance apparatus).

In one embodiment, a deflection of the magnetization of a nuclear spin is caused by the application (e.g., the transmission) of a radio frequency transmission pulse (e.g., of the first and/or second radio frequency transmission pulse). The greater the energy of the radio frequency transmission pulse is, the stronger the deflection may be. The final deflection of the magnetization after the end of the RF pulse may often also be referred to as the flip angle.

In one embodiment, the provision of the first radio frequency transmission pulse and the second radio frequency transmission pulse involves the first radio frequency transmission pulse being generated (e.g., calculated) while taking a first boundary condition into account and the second radio frequency transmission pulse being generated (e.g., calculated) while taking a second boundary condition into account. In this context, the first and the second boundary conditions relate to a maximum amount of energy that may be emitted by the respective radio frequency transmission pulse (e.g., an amount of energy that may be introduced into the patient and/or absorbed by the patient).

For example, the energy of the first radio frequency transmission pulse is greater than the energy of the second radio frequency transmission pulse. In one embodiment, the energy input into the patient by the second radio frequency transmission pulse (e.g., during scanning of the at least one second region) is smaller than the energy input into the patient by the first radio frequency transmission pulse (e.g., during scanning of the at least one first region).

In one embodiment, the first boundary condition allows for a higher amount of energy than the second boundary condition. In one embodiment, allowing for a higher amount of energy may improve the quality of the image data generated by the first radio frequency transmission pulse. In one embodiment, a lower amount of energy results in lower SAR exposure for the patient.

For example, a first amount of energy is determined to generate the first radio frequency transmission pulse. For example, a second amount of energy is determined to generate the second radio frequency transmission pulse. In one embodiment, the first amount of energy is greater than the second amount of energy. For example, the first amount of energy is greater than the second amount of energy by a factor of at least 1.2, 1.5, 2, or 3.

In one embodiment, the generation of the first radio frequency transmission pulse involves optimizing the first radio frequency transmission pulse while taking into account the first boundary condition. In one embodiment, the generation of the second radio frequency transmission pulse involves optimizing the first radio frequency transmission pulse while taking into account the second boundary condition. For example, optimizing the first radio frequency transmission pulse and/or the second radio frequency transmission pulse involves optimizing the image quality achievable by the radio frequency transmission pulses.

In one embodiment, the provision of the first radio frequency transmission pulse and/or the second radio frequency transmission pulse involves optimizing the first radio frequency transmission pulse and/or the second radio frequency transmission pulse, a cost function being minimized in the process. The cost function includes, as a first optimization parameter, the homogeneity of a B1 field distribution and/or flip angle distribution to be generated by the respective radio frequency transmission pulse, and, as a second optimization parameter, an amount of energy that may be emitted by the respective radio frequency transmission pulse (e.g., an amount that may be introduced into the patient and/or absorbed by the patient).

In one embodiment, the cost function has a weighting of the first optimization parameter relative to the second optimization parameter. In one embodiment, the weighting differentiates in the optimization of the first radio frequency transmission pulse compared to the optimization of the second radio frequency transmission pulse.

In one embodiment, the weighting is such that, when optimizing the first radio frequency transmission pulse, a high level of homogeneity of a B1 field distribution and/or flip angle distribution to be generated is more important (e.g., has a greater impact on the value of the cost function) than when optimizing the second radio frequency transmission pulse. In one embodiment, the weighting is such that, when optimizing the second radio frequency transmission pulse, the smallest amount of energy that may be emitted (e.g., the amount of energy that may be introduced into the patient and/or absorbed by the patient) is more important (e.g., has a greater impact on the value of the cost function) than when optimizing the first radio frequency transmission pulse.

In one embodiment, when optimizing the first radio frequency transmission pulse, the highest possible homogeneity of a B1 field distribution and/or flip angle distribution to be generated is given greater weight than the lowest possible amount of energy. In one embodiment, when optimizing the second radio frequency transmission pulse, the lowest possible amount of energy is given greater weight than the highest possible homogeneity of the B1 field distribution and/or flip angle distribution to be generated.

In one embodiment, the first magnetic resonance signals are suitable for generating (e.g., reconstructing) high-quality image data. In one embodiment, the first radio frequency transmission pulse is optimized for generating the first magnetic resonance signals in order to produce the best possible image data. For example, it may be accepted that other aspects of the use of the first radio frequency transmission pulse are suboptimal (e.g., patient exposure as a result of irradiation of the first radio frequency transmission pulse into the patient) and/or suffer in terms of image quality as a result of optimization.

In one embodiment, the first magnetic resonance signals are suitable for generating first image data, and the second magnetic resonance signals are suitable for generating second image data, the first image data having a higher quality (e.g., image quality) than the second image data. In one embodiment, a magnetic resonance image that is generated from image data of higher quality (e.g., image quality) exhibits a lower degree of image artifacts (e.g., ghosting) and/or higher image sharpness.

In one embodiment, the first radio frequency transmission pulse produces a B1 field distribution that has a lower coefficient of variation (COV) than the B1 field distribution that is produced by the second radio frequency transmission pulse. Depending on the part of the body to be scanned or measured, the transmitter coil used, the magnetic resonance sequence used, etc., an advantageous coefficient of variation may vary in magnitude. For example, in a brain measurement, an advantageous coefficient of variation for the first radio frequency transmission pulse may be a value of less than 20%, and an advantageous coefficient of variation for the second radio frequency transmission pulse may be a value of less than 50%.

In one embodiment, the first radio frequency transmission pulse is optimized to produce a homogeneous B1 field distribution and/or homogeneous flip angles. In one embodiment, the method includes optimization of a radio frequency transmission pulse for the provision of the first radio frequency transmission pulse.

In one embodiment, the first radio frequency transmission pulse is a dynamic pulse (e.g., a pTx pulse). In one embodiment, such pulses are suitable for optimization for the generation of a homogeneous B1 field distribution and/or homogeneous flip angles.

For example, a radio frequency transmission pulse, the phase and/or amplitude of which changes over the course of the pulse, may be regarded as a dynamic pulse. For example, a gradient coil unit of the magnetic resonance apparatus may be used in the meantime to scan a gradient trajectory (e.g., a predetermined gradient trajectory).

For example, the scanning of the gradient trajectory occurs in time with the variation of the phase and/or amplitude of the radio frequency transmission pulse.

For example, the dynamic pulse has an arbitrary pulse shape. In one embodiment, the second radio frequency transmission pulse has a predetermined pulse shape (e.g., not an arbitrary pulse shape).

In one embodiment, at least one magnetic field map is determined. In one embodiment, the at least one magnetic field map includes at least one B1 field map, or B1 map, for short, and/or at least one B0 field map, or B0 map, for short. For example, the pulse shape of the at least one dynamic pulse is determined based on the at least one magnetic field map. In one embodiment, the second radio frequency transmission pulse is independent of the at least one magnetic field map.

A possible embodiment of the method provides that the determination of the at least one dynamic pulse includes the optimization of the homogeneity of a B1 field distribution and/or flip angle distribution that may be generated, and, for example, is to be generated, by the dynamic pulse. In one embodiment, the optimization is performed based on the at least one magnetic field map. In one embodiment, the dynamic pulse (e.g., its pulse shape) is optimized based on the at least one magnetic field map (e.g., with regard to the maximum homogeneity of the B1 field distribution and/or flip angle distribution). In one embodiment, any pulse shape is available for the optimization of the B1 field distribution and/or flip angle distribution.

For example, a pulse shape is the profile and/or the form of a pulse. A pulse shape may be described, for example, by an envelope of the pulse (e.g., in the base band). The pulse shape is, for example, independent of an absolute height (e.g., amplitude) and/or an absolute width (e.g., length) of the pulse.

A pulse shape of a pulse may be described, for example, by the chronological course of the pulse (e.g., of an instantaneous value and/or a deflection of the pulse; expressed as a pulse profile S(t), it being possible to describe the pulse profile S(t) using a complex-valued function and t representing the time).

The at least one pulse shape and/or amplitude and/or phase of the RF transmission pulse or a sub-pulse may, for example, correspond to a shape and/or amplitude and/or phase of a voltage pulse that is applied to the respective transmitter coil, and/or a current pulse that flows through the transmitter coil.

The at least one pulse shape and/or amplitude and/or phase of the gradient pulse may, for example, correspond to a shape and/or amplitude and/or phase of a voltage pulse that is applied to the gradient coil unit, and/or a current pulse that flows through the gradient coil unit.

A dynamic pulse may enable more precise control of the resulting B1 field. For example, a pTx pulse may be used to compensate for magnetic field inhomogeneities that, above all, may be advantageous at higher field strengths of the main magnetic field. Dynamic parallel transmission may be advantageous at high field strengths because the B1 effects that occur there may be of such a shortwave nature that they would be visible within a magnetic resonance image. However, as described above, the use of a dynamic pulse may also be advantageous at lower field strengths, such as, for example, for fat saturation. In one embodiment, the dynamic pulse is a fat saturation pulse.

A possible embodiment of the method provides that the first radio frequency transmission pulse is a pTx pulse. “pTx” stands for “parallel transmission”. A pTx pulse may, for example, include a number of sub-pulses that are transmitted in parallel (e.g., simultaneously) by one transmitter coil each of a radio frequency antenna unit of the magnetic resonance apparatus. For example, a transmitting channel may be assigned to each transmitter coil. The sub-pulses may differ (e.g., in their pulse shape and/or amplitude and/or phase). Further, the sub-pulses may, for example, exhibit a time lag relative to one another. For example, a first radio frequency transmission pulse consists of several sub-pulses that differ from one another and may each be transmitted by a transmitter coil of a multi-channel transmitter coil arrangement of the radio frequency antenna unit. In one embodiment, at least some of the multiple sub-pulses (e.g., all of the sub-pulses) are dynamic pulses.

When transmitting a pTx pulse, a predetermined spatial distribution of the excitation may be achieved as an additional degree of freedom through interference of the signals from a plurality of transmitting channels via a plurality of transmitter coils of the radio frequency antenna unit that is adjusted when determining the pTx pulse (e.g., by varying the phase and amplitude).

The B1 field map may describe the local distribution of a B1 field. A B1 map may be a representation, (e.g., a spatial representation) of a B1 field. The B1 field may be an alternating magnetic field (e.g., from RF radiation) that may be generated by a radio frequency antenna unit of a magnetic resonance apparatus. The B0 field map may describe the spatial distribution of a B0 field. A B1 map may be a representation (e.g., a spatial representation) of a B0 field. The B0 field may be the static main magnetic field of the magnetic resonance apparatus. The B0 field may be generated by a main magnet of the magnetic resonance apparatus, which is often superconducting. A B1 field or a B0 field may, for example, be represented by an image in which the intensity of an image pixel or image voxel represents the strength of the B1 field or the B0 field at the location of the image pixel or image voxel.

In one embodiment, the B1 and/or the B0 field describe the respective field distribution when the patient is located in an examination area of the magnetic resonance apparatus (e.g., in a measurement position). For example, at higher magnetic field strengths, the presence of the patient may cause significant inhomogeneities of the fields. In one embodiment, such inhomogeneities may be reduced by the first radio frequency transmission pulse.

In one embodiment, the second radio frequency transmission pulse is configured to cause (only) a slight (e.g., as slight as possible) exposure of the patient during the excitation of the second magnetic resonance signals.

In one embodiment, the second radio frequency transmission pulse is configured to produce lower exposure of the patient during the excitation of the second magnetic resonance signals than during the excitation of the second magnetic resonance signals by the first radio frequency transmission pulse.

For example, the aforementioned exposure of the patient involves the absorption of energy from the radio frequency transmission pulse by the patient. For example, the aforementioned exposure of the patient involves SAR exposure of the patient.

For example, the aforementioned exposure of the patient involves nerve stimulation (e.g., peripheral nerve stimulation (PNS)) of the patient.

Time-varying magnetic fields caused by radio frequency transmission pulses may affect the human body such that the electric fields generated thereby induce electric currents in the body of the patient and nerves or muscles of the patient are stimulated. This stimulation may be perceived as unpleasant by the patient.

In one embodiment, a compromise may be found by using an image-optimized first radio frequency transmission pulse and an exposure-optimized second radio frequency transmission pulse that enables scanning of the k-space with acceptable patient exposure from which good magnetic resonance images may still result.

In one embodiment, the second radio frequency pulse is a circular polarization (CP). In one embodiment, circular polarizations result in relatively low SAR exposure. In one embodiment, circular polarizations exhibit high (e.g., physical) efficiency. In one embodiment, circular polarizations are particularly easy to calculate and/or simulate and/or optimize.

In one embodiment, the at least one first region includes a first portion of the k-space. The first portion of the k-space includes more than 50% or more than 60% of the (entire) k-space. In one embodiment, the at least one second region includes a second portion of the k-space. In one embodiment, the sum of the first portion and the second portion of k-space is 100 % of the k-space.

For example, it has been shown that up to 40% of the k-space may be scanned using low-power (e.g., circularly polarized) second radio frequency transmission pulses without resulting in a significant loss of image quality.

In one embodiment, the k-space includes the at least one first region and the at least one second region. In one embodiment, only two radio frequency transmission pulses are then provided (e.g., the first radio frequency transmission pulse and the second radio frequency transmission pulse). The provision of radio frequency transmission pulses therefore requires little effort.

However, it is also conceivable that in addition to the at least one first region and the at least one second region, the k-space is segmented into further regions (e.g., at least one third region, at least one fourth region, etc.). These regions may be scanned using additional radio frequency transmission pulses (e.g., the at least one third region using a third radio frequency transmission pulse, the at least one fourth region using a fourth radio frequency transmission pulse, etc.).

In one embodiment, the at least one first region and/or the at least one second region is determined by a simulation and/or a neural network.

For example, a simulation and/or a neural network may be used to calculate a scan of the at least one first region using the first radio frequency transmission pulse and/or a scan of the at least one second region using the second radio frequency transmission pulse. In one embodiment, the at least one first region and/or the at least one second region may be determined based on such a calculation.

Such a simulation may, for example, be performed using an Extended Phase Graph (EPG) simulation (e.g., an Extended Phase Graph simulation informed by k-space). Such a simulation may, for example, be performed using a technology referred to as MRzero, which is described in the publication A. Loktyushin et al., MRzero—Fully automated discovery of MRI sequences using supervised learning, arXiv: 2002.04265.

Training of a neural network to determine the at least one first region and/or the at least one second region may, for example, include an entire k-space being scanned twice: i) using a first radio frequency transmission pulse (for the purpose of the training); and ii) using a second radio frequency transmission pulse (for the purpose of the training).

Thereupon, the neural network may determine for each element of the k-space (e.g., a line, such as a row, of the k-space), based on a loss criterion, whether the element is scanned using the first radio frequency transmission pulse or using the second radio frequency transmission pulse. The loss criterion may include two parts. A first of the two parts is a) the quality of a resulting magnetic resonance image. The quality may, for example, be assessed based on a comparison of the resulting magnetic resonance image with a magnetic resonance image (e.g., optimized for image quality) in which only first radio frequency transmission pulses are used. A second of the two parts is b) patient exposure resulting from the application of the radio frequency transmission pulses. For example, an attempt may be made to minimize the number of second radio frequency transmission pulses in order, for example, to minimize SAR exposure.

Further, a magnetic resonance apparatus is proposed that is configured to execute a computer-implemented method as described above for scanning a k-space during a magnetic resonance measurement of a patient. In one embodiment, the magnetic resonance apparatus includes a computing unit, for example, including one or more processors and/or a memory unit (e.g., including one or more memory modules) in order to execute the method.

Further, a computer program product that includes a program and may be loaded directly into the memory of a programmable system control unit of a magnetic resonance apparatus is provided. The computer program product includes program means (e.g., libraries and help functions) for executing a proposed method for scanning a k-space during a magnetic resonance measurement of a patient when the computer program product is executed in the system control unit of the magnetic resonance apparatus. The computer program product may include software with source code that still needs to be compiled and linked, or which only needs to be interpreted, or executable software code that only needs to be loaded into the system control unit for execution.

The computer program product enables the proposed method to be executed advantageously quickly, repeated identically, and in a robust manner. The computer program product may be configured such that the computer program product may execute the proposed method acts using the system control unit. The system control unit has the necessary prerequisites, such as, for example, an appropriate working memory, a corresponding graphics card, or a corresponding logic unit, so that the respective method acts may be executed efficiently.

For example, the computer program product is stored on a computer-readable medium (e.g., a non-transitory computer-readable storage medium) or hosted on a network or server, from where it may be loaded into the processor of a local system control unit that may be directly connected to the magnetic resonance apparatus or may be configured as part of the magnetic resonance apparatus. Further, control information of the computer program product may be stored on an electronically readable data carrier. The control information of the electronically readable data carrier may be configured such that, when the data carrier is used in a system control unit of a magnetic resonance apparatus, it executes a proposed method.

Examples of electronically readable data carriers are a DVD, a magnetic tape, or a USB stick on which electronically readable control information (e.g., software) is stored. When this control information is read from the data carrier and stored in a system control unit of the magnetic resonance apparatus, all the proposed embodiments of the previously described method may be implemented.

Further advantages, features, and details of the invention will emerge from the example embodiments described hereinafter and with reference to the drawings. Corresponding parts are labeled with the same reference characters in all the figures.

### BRIEF DESCRIPTION OF THE DRAWINGS

FIG. 1 is diagrammatic view of a magnetic resonance apparatus;

FIG. 2 illustrates a sequence of a process for scanning a k-space during a magnetic resonance measurement of a patient;

FIG. 3 show a number of scanning patterns of a k-space with varying numbers of first and second regions;

FIG. 4 illustrates an image quality measure as a function of a ratio of a size of regions of a k-space scanned using two different radio frequency transmission pulses; and

FIG. 5 illustrates a sequence for generating a load-specific segmentation of k-space.

### DETAILED DESCRIPTION

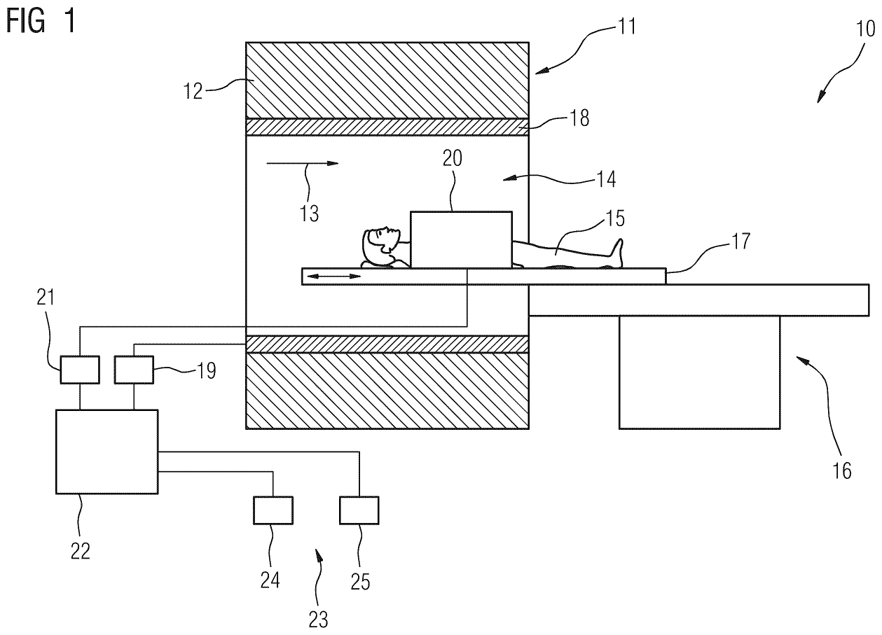

FIG. 1 shows a diagrammatic view of a magnetic resonance apparatus 10. The magnetic resonance apparatus 10 includes a magnet unit 11 that has a main magnet 12 for generating a strong and, for example, temporally constant main magnetic field 13 (B0 field). In addition, the magnetic resonance apparatus 10 includes a patient receiving area 14 for recording a patient 15. In the present example embodiment, the patient receiving area 14 is cylindrical in design and is cylindrically surrounded by the magnet unit 11 in a circumferential direction. In principle, however, an embodiment of the patient receiving area 14 deviating therefrom may be provided at any time. The patient 15 may be moved into the patient receiving area 14 by a patient positioning apparatus 16 of the magnetic resonance apparatus 10. The patient positioning apparatus 16 includes a patient table 17 that is configured to be movable inside the patient receiving area 14.

The magnet unit 11 also includes a gradient coil unit 18 for generating magnetic field gradients that are used for spatial encoding during imaging. The gradient coil unit 18 is controlled using a gradient control unit 19 of the magnetic resonance apparatus 10. The magnet unit 11 also includes a radio frequency antenna unit 20 that is configured as a local coil in the present example embodiment. However, for example, it is also conceivable that the radio frequency antenna unit 20 is configured as a body coil that is permanently integrated into the magnetic resonance apparatus 10. The radio frequency antenna unit 20 may, for example, have a number of transmission antennae, so that parallel transmission technology (pTx) is made possible. The radio frequency antenna unit 20 is controlled by a radio frequency antenna control unit 21 of the magnetic resonance apparatus 10 and emits radio frequency transmission pulses for the generation of a B1 field into an examination area that is essentially formed by a patient receiving area 14 of the magnetic resonance apparatus 10. This results in the excitation of atomic nuclei by the main magnetic field 13 generated by the main magnet 12. Magnetic resonance signals are generated by the relaxation of the excited atomic nuclei. The radio frequency antenna unit 20 is also configured to receive the magnetic resonance signals.

The magnetic resonance apparatus 10 has a system control unit 22 for controlling the main magnet 12 and the gradient control unit 19, and for controlling the radio frequency antenna control unit 21. The system control unit 22 centrally controls the magnetic resonance apparatus 10, for example, by performing a magnetic resonance sequence. When performing the magnetic resonance sequence, k-space may be scanned (e.g., using a sequence of sequence segments, each of which includes a radio frequency transmission pulse). For example, the system control unit 22 may output a signal that is amplified by a radio frequency amplifier (not explicitly shown) and is emitted by the radio frequency antenna unit 20.

In addition, the system control unit 22 includes an evaluation unit, not shown in detail, for evaluating the magnetic resonance signals that are acquired during the magnetic resonance examination. Further, the magnetic resonance apparatus 10 includes a user interface 23 that is connected to the system control unit 22. Control information such as, for example, imaging parameters, and reconstructed magnetic resonance images may be displayed on a display unit 24 (e.g., on at least one monitor) of the user interface 23 for medical operating personnel. Further, the user interface 23 has an input unit 25 by which information and/or parameters may be entered by medical operating personnel during a measurement process.

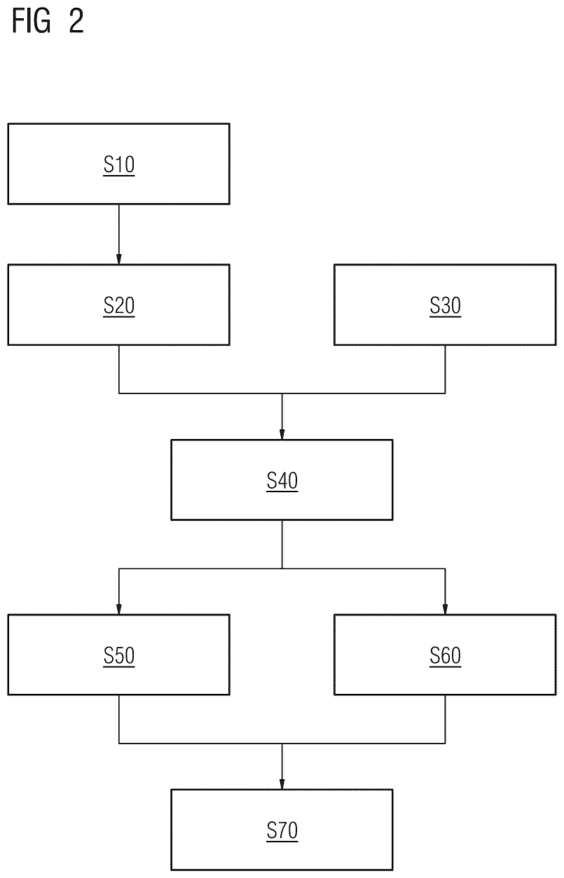

FIG. 2 illustrates a computer-implemented method for scanning k-space during magnetic resonance measurement of a patient 15. In act S10, at least one magnetic field map (e.g., a B1 field map and/or a B0 field map) is determined. In act S20, a first radio frequency transmission pulse for generating first magnetic resonance signals is determined and provided by the at least one magnetic field map.

In one embodiment, the first magnetic resonance signals are suitable for generating high-quality image data. In one embodiment, the first radio frequency transmission pulse is optimized to generate homogeneous B1 field distribution and/or homogeneous flip angle distribution. For example, dynamic pulses and/or pTx pulses are suitable as first radio frequency transmission pulses.

In act S30, a second radio frequency transmission pulse for generating second magnetic resonance signals is provided. In one embodiment, the second radio frequency transmission pulse is configured to cause minimal discomfort to the patient 15 during the excitation of the second magnetic resonance signals. This exposure of the patient 15 may include, for example, absorption of energy from the radio frequency transmission pulse by the patient 15 (e.g., SAR exposure) and/or nerve stimulation of the patient 15. The second radio frequency transmission pulse may, for example, be a circularly polarized pulse.

The SAR exposure of a radio frequency transmission pulse describes the amount of energy that the radio frequency transmission pulse applies to the patient 15. Only a specified SAR exposure limit may be permitted in order to ensure the safety of the patient. pTx radio frequency transmission pulses may generate a homogeneous B1 field, making them suitable for the generation of high-quality image data. pTx radio frequency transmission pulses use multiple transmission pulses from the radio frequency antenna unit 20 in parallel. As a result of this, a pTx pulse may have a high energy content. However, this may lead to increased SAR exposure for the patient 15. In contrast, the second radio frequency transmission pulse may only have a low energy content.

In act S40, the k-space to be scanned is segmented into at least one first region and at least one second region. In act S50, the at least one first region of the k-space is scanned using the first radio frequency transmission pulse. In act S60, the at least one second region of the k-space is scanned using the second radio frequency transmission pulse. Based on the magnetic resonance signals acquired through this scanning, at least one magnetic resonance image is reconstructed in S70.

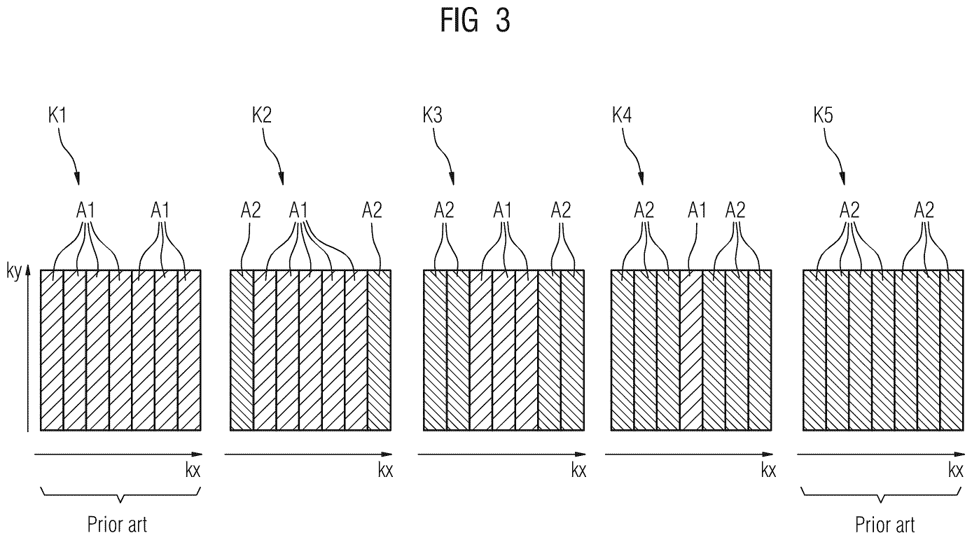

The segmentation of k-space in act S40 and the scanning of k-space in act S50 and act S60 will be explained by way of example with reference to FIG. 3. Various k-spaces K1 through K5 are shown there. The k-spaces here are divided into seven regions that lie adjacent to one another along the kx axis.

For example, such regions may be scanned using a RESOLVE sequence (e.g., Readout Segmentation Of Long Variable Echo-trains). In this approach, the readout of the magnetic resonance signals is segmented into a number of shorter echo trains. Instead of capturing all k-space lines in a single acquisition (e.g., as is the case with customary single-shot EPI), RESOLVE divides the scan into a number of regions, each with fewer k-space lines. This reduces the echo interval and the echo train length and minimizes the distortions caused by susceptibility.

The k-space K1 only has first regions A1 that are intended to be sampled using the first radio frequency transmission pulse (e.g., the entire k-space is scanned using only the first radio frequency transmission pulse). The k-space K5 only has second regions A2 that are intended to be sampled using the second radio frequency transmission pulse (e.g., the entire k-space is scanned using only the second radio frequency transmission pulse). Such k-space scans are known from the prior art. The scanning of k-space K1 provides image data from which magnetic resonance images may be reconstructed with particularly high image quality. The scanning of k-space K5 is particularly gentle on the patient 15 (e.g., the exposure of the patient is particularly low).

Instead of applying the first radio frequency transmission pulse (which provides good image data) to every k-space line, it is proposed to apply the first radio frequency transmission pulse only to a part thereof and to use the second radio frequency transmission pulse (which is gentler on the patient) for the other lines. As a result, only a fraction of the patient exposure (e.g., SAR exposure) is required, and the image quality is largely preserved. One possible reason for this is that, although a k-space line is acquired using the second radio frequency transmission pulse (e.g., during the scanning of a second region), the preceding good magnetization from a first radio frequency transmission pulse (e.g., during the scanning of a first region) is still present.

When scanning k-space K2, compared to the scanning of k-space K1, the first and seventh segments are scanned using the second radio frequency transmission pulse (e.g., these segments are second regions). For example, this enables the SAR exposure to be reduced.

The SAR exposure may be reduced even further if additional segments of the k-space are scanned using the second radio frequency transmission pulse, as illustrated for k-spaces K3 and K4. Thus, in k-space K3, the two outer segments on each side are referred to as second regions A2. In k-space K4, only the middle segment remains as the first region A1.

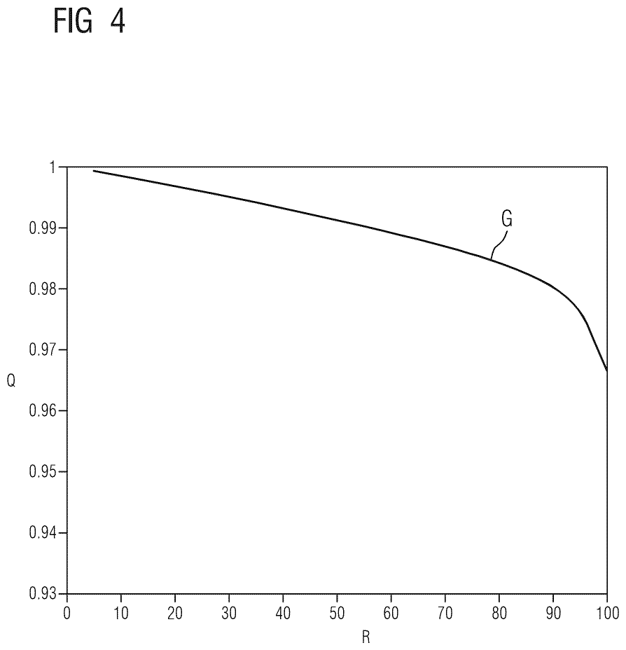

FIG. 4 shows a quantitative representation of the dependence of an image quality measure Q of a magnetic resonance image on a proportion R of k-space regions that are scanned using a second radio frequency transmission pulse (e.g., a CP pulse). In one embodiment, the image quality measure Q indicates a structural similarity index measure (SSIM) that a magnetic resonance image exhibits relative to a reference magnetic resonance image. The reference magnetic resonance image is the magnetic resonance image that results from a reconstruction of exclusively first magnetic resonance signals (e.g., from magnetic resonance signals from a scan in which the k-space was scanned exclusively using the (high-quality) first radio frequency transmission pulse (a pTx pulse); this corresponds to the portion R=0).

If R is now increased (e.g., if more and more k-space elements are scanned using the second radio frequency transmission pulse), the similarity between the resulting magnetic resonance image and the reference magnetic resonance image decreases (e.g., the image quality of the resulting magnetic resonance image tends to deteriorate, but initially (at low R) only to a very small extent). This is reflected in the fact that the curve G is very flat at low R values. In one embodiment, R<50% (e.g., <40% or <30%). At such levels, the image quality is still very high (e.g., Q>0.99 for R<40%) while already achieving a significant reduction in SAR exposure.

It should also be noted that it is also conceivable to regard two adjacent segments that are scanned using the same radio frequency transmission pulse as a first region and a second region, respectively. Thus, for example, the two outer segments in k-space K3 in FIG. 3 may be regarded as a second region.

In principle, any distribution of first and second regions in k-space may be provided. Any number of first and second regions may also be provided. The provision of additional radio frequency transmission pulses (e.g., third, fourth, etc. radio frequency transmission pulses) for additional regions (e.g., third, fourth, etc. regions) may also be provided.

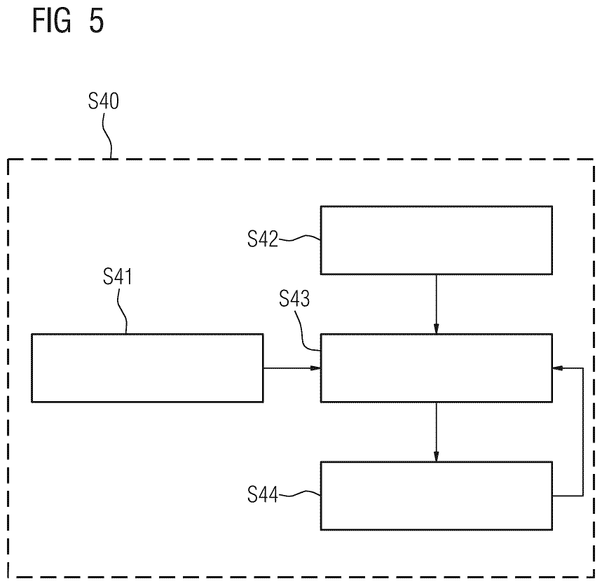

FIG. 5 shows a possible embodiment for segmenting the k-space to be scanned into at least one first region and at least one second region. In act S41, an exposure limit (e.g., an SAR limit) is provided. In act S42, an initial k-space segmentation is provided, the entire k-space being formed by an initial first region. According to this initial k-space segmentation, it is therefore provided that the entire k-space is scanned using the first radio frequency transmission pulse. In act S43, it is verified whether scanning of the k-space according to the initial k-space segmentation would result in the exposure limit provided in act S41 being exceeded. If this is the case, in act S44, an updated segmentation of the k-space is performed, in which at least one second region is enlarged (e.g., according to this updated segmentation of the k-space, it is intended to scan a larger region of the k-space using the second radio frequency transmission pulse). An enlargement of the at least one second region may, for example, include the enlargement of existing second regions and/or the creation of new second regions. Based on the initial k-space-segmentation, the second region (e.g., which initially does not yet exist) is thus first created. Then, in an iteration in act S43, it is verified again whether scanning the k-space according to the updated k-space segmentation would result in exceeding the exposure limit provided in act S41. If this is not the case, an updated segmentation of the k-space is performed again in act S44, in which the at least one second region is (e.g., further) enlarged. The iterative acts S43 and S44 are performed until the exposure limit is no longer exceeded. The resulting k-space segmentation is provided for the possible subsequent acts S50 and S60 (e.g., the k-space is scanned in acts S50 and S60 according to this segmentation).

Finally, it should be noted once again that the methods described in detail above, as well as the magnetic resonance apparatus illustrated, are merely example embodiments that may be modified by a person skilled in the art without departing from the scope of the invention. Further, the use of the indefinite articles “a” or “an” does not preclude the possibility that the relevant features may be present multiple times. Similarly, the term “unit” does not preclude the possibility that the relevant components consist of a number of interacting subcomponents, which may also be spatially distributed. Independent of the grammatical term usage, individuals with male, female, or other gender identities are included within the term.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for scanning a k-space during a magnetic resonance measurement of a patient, the method being computer-implemented and comprising:
providing a first radio frequency transmission pulse for generating first magnetic resonance signals;
providing a second radio frequency transmission pulse for generating second magnetic resonance signals;
segmenting the k-space to be scanned in at least one first region and at least one second region;
scanning the at least one first region of the k-space using the first radio frequency transmission pulse; and
scanning the at least one second region of the k-space using the second radio frequency transmission pulse.

2. The method of claim 1, wherein providing the first radio frequency transmission pulse and the second radio frequency transmission pulse comprise:
generating the first radio frequency transmission pulse while taking into account a first boundary condition; and
generating the second radio frequency transmission pulse while taking into account a second boundary condition,
wherein the first boundary condition and the second boundary condition relate to a maximum amount of energy that is emittable by the respective radio frequency transmission pulse.

3. The method of claim 2, wherein the first boundary condition and the second boundary condition relate to an amount of energy that is introducible into the patient, absorbed by the patient, or introducible into the patient and absorbed by the patient.

4. The method of claim 2, wherein the first boundary condition allows for a greater amount of energy than the second boundary condition.

5. The method of claim 1, wherein providing the first radio frequency transmission pulse, the second radio frequency transmission pulse, or the first radio frequency transmission pulse and the second radio frequency transmission pulse comprises:
optimizing the first radio frequency transmission pulse, the second radio frequency transmission pulse, or the first radio frequency transmission pulse and the second radio frequency transmission pulse, wherein the optimizing of the first radio frequency transmission pulse, the second radio frequency transmission pulse, or the first radio frequency transmission pulse comprises minimizing a cost function,
wherein the cost function includes, as optimization parameters, homogeneity of a B1 field distribution, a flip angle distribution to be generated by the respective radio frequency transmission pulse, or the homogeneity and the flip angle distribution, and
wherein the cost function includes, as optimization parameters, an amount of energy that is emittable by the respective radio frequency transmission pulse.

6. The method of claim 5, wherein the cost function includes, an optimization parameters, an amount of energy that is introducible into the patient, absorbed by the patient, or introducible into the patient and absorbed by the patient.

7. The method of claim 1, wherein the first magnetic resonance signals are suitable for generating high-quality image data.

8. The method of claim 1, wherein the first radio frequency transmission pulse is optimized to generate a homogeneous B1 field distribution, a homogeneous flip angle distribution, or a homogeneous B1 field and homogeneous flip angle distribution.

9. The method of claim 1, wherein the first radio frequency transmission pulse is a dynamic pulse.

10. The method of claim 1, wherein the second radio frequency transmission pulse is configured to cause minimal discomfort to the patient during the excitation of the second magnetic resonance signals.

11. The method of claim 1, wherein exposure of the patient involves absorption of energy from the radio frequency transmission pulse by the patient.

12. The method of claim 1, wherein exposure of the patient involves nerve stimulation of the patient.

13. The method of claim 1, wherein the second radio frequency transmission pulse is a circular polarization.

14. The method of claim 1, wherein the at least one first region comprises a first portion of the k-space, and
wherein the first portion of the k-space comprises more than 50% of the k-space.

15. The method of claim 1, wherein the at least one first region, the at least one second region, or the at least one first region and the at least one second region is determined using a simulation, a neural network, or the simulation and the neural network.

16. A magnetic resonance apparatus comprising:
a processor configured to scan a k-space during a magnetic resonance measurement of a patient, the processor being configured to scan the k-space during the magnetic resonance measurement of the patient comprising the processor being configured to:
provide a first radio frequency transmission pulse for generating first magnetic resonance signals;
provide a second radio frequency transmission pulse for generating second magnetic resonance signals;
segment the k-space to be scanned in at least one first region and at least one second region;
scan the at least one first region of the k-space using the first radio frequency transmission pulse; and
scan the at least one second region of the k-space using the second radio frequency transmission pulse.

17. A non-transitory computer-readable storage medium that stores instructions executable by a programmable system control unit of a magnetic resonance apparatus to scan a k-space during a magnetic resonance measurement of a patient, the instructions comprising:
providing a first radio frequency transmission pulse for generating first magnetic resonance signals;
providing a second radio frequency transmission pulse for generating second magnetic resonance signals;
segmenting the k-space to be scanned in at least one first region and at least one second region;
scanning the at least one first region of the k-space using the first radio frequency transmission pulse; and
scanning the at least one second region of the k-space using the second radio frequency transmission pulse.
